(19) United States
(12) Patent Application Publication
(10) Pub. No.: US 2011/0288314 A1
(43) Pub. Date: Nov. 24, 2011

(54) PHLOROGLUCINOL DERIVATIVES FROM ECKLONIA CAVA HAVING ANTI-HIV-1 INHIBITORY ACTIVITY

(76) Inventors: Moon-Moo Kim, Busan (KR); Se-Kwon Kim, Busan (KR); Sang-Hoon Lee, Busan (KR)

(21) Appl. No.: 13/130,335

(22) PCT Filed: Dec. 9, 2008
(86) PCT No.: PCT/KR08/07276

§ 371 Date: Aug. 11, 2011

(30) Foreign Application Priority Data
Dec. 1, 2008 (KR) 10-2008-0120582

(51) Int. Cl. C07D 407/10

(52) U.S. Cl. 549/359

(57) ABSTRACT

The present invention relates to a pharmaceutical composition and health functional food having anti-HIV-1 inhibitory activity containing phloroglucinol derivative, 6,6′-bieckol as an effective ingredient, EC extract and its phloroglucinol derivative, 6,6′-bieckol according to the present invention does not show cytotoxic effect compared to other tannin, and have effects to inhibit the HIV-1 induced syncytia formation and the p24 antigen production as well as the HIV-1 reverse transcriptase activity.

### TECHNICAL FIELD

The present invention relates to phloroglucinol derivatives from Ecklonia cava having anti-HIV-1 inhibitory activity, more specifically to a pharmaceutical composition and health functional food having anti-HIV-1 inhibitory activity containing phloroglucinol derivative, 6,6′-bieckol as an effective ingredient.

### BACKGROUND ART

Human immunodeficiency virus type-1 (HIV-1) is identified as the causative agent of acquired immunodeficiency syndrome (AIDS) which is one of the most important diseases with about 33.2 million people infected worldwide up to now. In 2007, 2.1 million people died of AIDS related diseases and 2.5 million new HIV infections occurred. According to UNAIDS estimations, every day 6800 people become newly infected with HIV and 5700 people die of AIDS and related disorders. However, the pathway by which the virus depletes the immune system is not fully identified.

After the introduction of AIDS in early 1980s, the first generation anti-HIV drugs have been developed as the promising agents to cure AIDS patients. However, failure in anti-AIDS treatment is observed in more than 50% of the patients infected with HIV as a result of drug-resistant strains of virus. This fact increases the need for potential candidates showing higher inhibitory activity against various HIV strains, including the ones which are resistant to drugs currently used for antiretroviral therapy.

Recently, there are significant advances in rational drug design and highly active compounds can be synthesized. However, natural occurring products are still known as the richest source of bioactive leading compounds, as well as their derivatives. In this regard, natural products are great sources for the development of new generation anti-HIV drugs which are more effective with less side-effect.

So far, numerous compounds isolated from natural resources have been found to exhibit significant anti-HIV activity. Considerable evidence has emerged not only from the research and publications, but also from controlled clinical studies of natural product-derived substances as important leads for the development of antiviral drugs against viral infections caused by HIV. Many compounds of plant origin that inhibit HIV during various stages of its life cycle have been described including alkaloids, coumarins, carbohydrates, flavonoids, lignans, phenolics, quinines, phospholipids, terpenes and tannins. Unlike terrestrial natural products, which are known to be used for hundreds of years as traditional medicine sources, the serious research on marine natural products started in the middle of the last century.

Since the oceans covering more than 70% of the world, they represent an enormous reservoir for the discovery of novel therapeutic agents. Having such a big potential, natural product research has increasingly turned to marine natural products, and some of them are currently in clinical or preclinical evaluation. Marine organisms are among the leading sources of anti-HIV natural products. The antiviral properties of cyanovirin-N, an 11-kDa protein from cyanobacteria, were investigated and found that it irreversibly inactivates HIV through its high affinity to gp120. Various sulfated polysaccharides, most of which have high HIV inhibitory activity at the viral entry stage, have been isolated from marine algae. Dextran sulfate, carrageenans, galactan sulfate, and xylomannan are among the leading marine-based compounds with anti-HIV activity.

Tannins are naturally occurring water-soluble polyphenolic compounds, which are thought to show their HIV-1 inhibitory mode of action by inhibiting polymerase and ribonuclease activities of HIV-1 RT. The gallotannins, geranium, and corilagin inhibited HIV-1 replication in MT-4 cells. Geraniim effectively blocked viral entry and inhibited RT activity of HIV-1 at an IC₅₀ of 1.9 μM. Tannin derivatives 1,3,4-tri-O-galloylquinic acid and 3,5-di-O-galloylshikimic acid inhibited virus-cell interactions probably via binding tightly to virions, inactivating them and prevent infection. Phlorotannins are tannin derivatives composed of several phloroglucinol units linked to each other in different ways and mostly isolated from red and brown alga. Phlorotannins are reported to have antioxidant, antiinflammatory, antibacterial, and anti-MMP activities. Ahn et al. claimed that phloroglucinol derivatives 8,8′-bieckol and 8′,4′″-dieckol inhibited the activity of recombinant RT and protease of HIV-1 in vitro.

As a part of our continuous research to discover the bioactive natural products from marine sources, a series of phloroglucinol derivatives from marine brown alga EC were obtained and characterized by physicochemical and NMR spectroscopic methods. Among these isolated phlorotannins, some of them showed the modern inhibition activity against HIV-1.

Herein, we reported the anti-HIV-1 activity of phloroglucinol derivative 6,6′-bieckol for the first time. The results obtained from experiments indicated that 6,6′-bieckol inhibited the cytopathic effects of HIV-1 including HIV-1 induced syncytia formation, lytic effects, and viral p24 antigen production, as well as exhibited RT enzyme inhibitory and HIV-1 entry activity in addition to its other biological properties. Moreover, unlike most of other tannins, 6,6′-bieckol exhibited no cytotoxicity at concentrations which inhibited HIV-1 replication almost completely.

### DISCLOSURE OF INVENTION

### Technical Problem

The object of the present invention is to provide a new use of phloroglucinol derivatives from Ecklonia cava for anti-HIV-1 inhibitory activity.

The another object of the present invention is to provide a pharmaceutical composition and health functional food having anti-HIV-1 inhibitory activity containing phloroglucinol derivative, 6,6′-bieckol as an effective ingredient.

### Technical Solution

The above objects was achieved by exracting and refining phloroglucinol derivative from EC, treating H9 and H9/HIV-1IIIB cell line with the derivative, and confirming inhibitory effect of cell elute effect of HIV-1 and formation of cell fusion.

The present invention presents a pharmaceutical composition having anti-HIV-1 inhibitory activity, containing extracts from Ecklonia cava as an effective ingredient.

The extract from Ecklonia cava according to the present invention is preferably EtOAc extract.

The present invention presents a pharmaceutical composition having anti-HIV-1 inhibitory activity, containing phloroglucinol derivatives from Ecklonia cava as an effective ingredient.

The phloroglucinol derivative according to the present invention is preferably 6,6′-bieckol of formula 1.

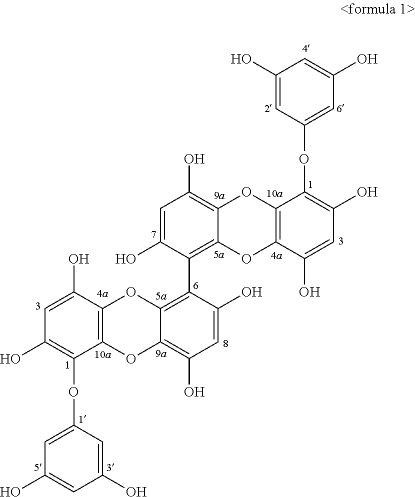

In the present invention, “effective ingredient” is meant that material or group of materials (comprising natural medicines having not revealed its pharmaceutically active components) which is expected to directly or indirectly express efficacy or effect by its inherented medicinal action, is comprised as a main ingredient.

The pharmaceutical composition of the present invention can further comprise appropriate carrier, excipient and diluent which are generally used in preparing pharmaceutical composition.

The pharmaceutical composition of the present invention can be administrated in the form of its pharmaceutically acceptable salt, and also can be administrated solely, or bind or combination with other pharmacetical active compound.

In addition, the present invention presents a health functional food having anti-HIV-1 inhibitory activity, containing extracts from Ecklonia cava as an effective ingredient.

The extract from Ecklonia cava according to the present invention is preferably EtOAc extract.

The present invention also presents a health functional food having anti-HIV-1 inhibitory activity, containing phloroglucinol derivatives from Ecklonia cava as an effective ingredient.

The phloroglucinol derivative according to the present invention is preferably 6,6′-bieckol of the above formula 1.

In the present invention, “health functional food” is meant a food which is prepared and processed with raw material or ingredient having useful functionality to a body according to Act for health functional food No. 6727, and “functional food” is meant a food which is taken with the purpose of controlling neutrients in the structure and function of a body, or obtaining useful effects in health including a physiological function.

The health functional food according to the present invention comprises the extract in amount of 0.01 to 95 wt %, preferably 1 to 80 wt % based on total amount.

Also, with the purpose of anti-HIV-1 inhibitory activity, the health functional food according to the present invention can be prepared and processed in the form of tablet, capsule, powder, granule, solution or pill.

In the preperation of EC extract, methanol extract was obtained from EC powders, suspended in water, and fractionized in order with n-hexane, CH₂Cl₂, EtOAc and n-BuOH. EtOAc fraction showed the most potent anti-HIV-1 activity to H9 and H9/HIV-1IIIB human cells. Ten fractions (F110) were obtained by treating the EtOAc fraction with silica gel column chromatography, and F5 fraction having the most potent anti-HIV-1 activity was purified to obtain phloroglucinol derivative, compound 1 of the formula 1 (102.85 mg). Molecular formula of compound 1 is C₃₆H₂₂O₁₈. On the basis of the above data and an exact comparison with those values in the literature, compound 1 was assigned to be 6,6′-bieckol.

In the result of the investigation of the anti-HIV-1 activity of phloroglucinol derivative, 6,6′-bieckol according to the present invention, it inhibited the HIV-1 induced syncytia formation in a dose-dependent manner, protected the cells from lytic effect of virus, potently inhibited the HIV-1 RT enzyme from cell culture supernatant and the HIV-1 entry. In addition 6,6′-bieckol inhibited cellvirus (FIG. 1B) and cell fusion (FIG. 3A).

6,6′-bieckol according to the present invention was shown to inhibit the cytopathic effects of HIV-1 on CEM-SS and C8166 cells as well as the activity of HIV-1 reverse transcriptase enzyme activity in vitro, and also inhibits the HIV-1 induced syncytia formation and the p24 antigen production.

6,6′-bieckol according to the present invention inhibited the p24 antigen production in a dose-dependent manner both in culture supernatant and cell (FIGS. 2A and B) by inhibiting the activity of reverse transcriptase.

In conclusion, 6,6′-bieckol is a novel safe compound of natural origin with significant HIV-1 replication and reverse transcriptase inhibitory activity. The present study suggests that 6,6′-bieckol might be a promising candidate for the design of novel HIV-1 RT inhibitors with its special structure.

### Advantageous Effects

As described above, EC extract and its phloroglucinol derivative, 6,6′-bieckol does not show cytotoxic effect compared to other tannin, and have effects to inhibit the HIV-1 induced syncytia formation and the p24 antigen production as well as the HIV-1 reverse transcriptase activity.

### BRIEF DESCRIPTION OF DRAWINGS

FIG. 1 is to show the cytotoxic effect and the HIV-1 inhibitory effect of 6,6′-bieckol according to the present invention.

FIG. 2 is to show the effect of 6,6′-bieckol to virial p24 protein production in the HIV-1 infected cells.

FIG. 3 is to show the effect of 6,6′-bieckol to the HIV-1 entry and the HIV-1 RT inhibitory effect.

### MODE FOR THE INVENTION

In the followings, the present invention will be illustrated with reference to examples and experimental examples. However, the present invention is not restricted by such examples.

### EXAMPLE

¹H NMR (400 MHz) and ¹³C NMR (100 MHz) spectra were recorded on a JEOL JNM-ECP 400 NMR spectrometer (JEOL, Japan), using DMSO-d₆ solvent peak (d 2.50 ppm in 1H and d 39.5 ppm in ¹³C NMR) as an internal reference standard. For some signals, the approximated third decimal place. This is to distinguish between signals of very close value but which could nevertheless be clearly differentiated by visual inspection of the spectra. MS spectra were obtained on a JEOL JMS-700 spectrometer (JEOL, Japan). Extraction of EC was performed using Extraction Unit (Dongwon Scientific Co., Korea). Column chromatography was carried out by silica gel 60 (230-400 mesh, Merck, Germany), Sephadex LH-20 (Sigma, St. Louis, Mo., USA). TLC was run on precoated Merck Kieselgel 60 F254 plates (0.25 mm) and the spots on the TLC plate were detected under UV lamp (254 and 365 nm) using CHCl₃/MeOH/H₂O/acetic acid (65:25:4:3, v/v) as development solvent system, and Vanillin (H₂SO₄ was employed as the detecting agent for phenolic compounds. All the solvents for column chromatography was of a reagent grade from commercial sources.

AZT, dextran sulfate, and polyethylene glycol (Mw 8000 Da) were obtained from Sigma Chemical Co. (St. Louis, Mo., USA). Primary and secondary antibodies for Western blot were purchased from SantaCruz Biotechnology (CA, USA). HIV-1 p24 antigen capture ELISA kit was obtained from Perkin-Elmer Life Sciences (Boston, Mass., USA). Reverse transcriptase activity assay kit was purchased from InvitroGen (CA, USA). Cell culture medium (RPMI 1640), penicillin/streptomycin, fetal bovine serum (FBS), and other cell culture materials were obtained from Gibco BRL, Life Technology (NY, USA) and Sigma Chemical Co. (St. Louis, Mo., USA).

All experiments were carried out at least in triplicate and results are reported as means±standard deviation.

### Example 1

### Extraction and Purification of Phloroglucinol Derivative from EC

The marine edible brown seaweed, EC, was collected from Jeju Island coast of Korea during the period from October 2004 to March 2005. Fresh EC was washed three times with water to remove salt. The lyophilized EC was ground into powder before extraction. The dried EC powder (10 kg) was extracted by stirring extraction unit with MeOH (35 L) for 10 days. The methanol extract (273 g) was suspended in water and partitioned with n-hexane (35.92 g), CH₂Cl₂ (20.49 g), EtOAc (24.87 g) and n-BuOH (106 g), in sequence. The EtOAc fraction (24.87 g), which exhibited the most potent anti-HIV activity on H9 and H9/HIV-1IIIB human cells, was subjected to a silica gel flash chromatography and eluted with a gradient solvent system of Hexane/EtOAc/MeOH to yield ten subfractions (F110). The F5 (378.39 mg) with the highest activity on anti-HIV was further purified by Sephadex LH-20 with only MeOH to afford the phloroglucinol derivative, compound 1 of formula 1 (102.85 mg).

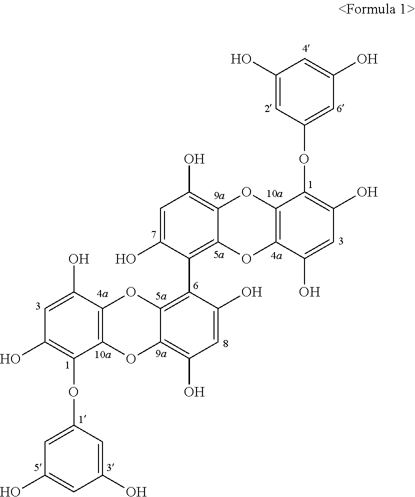

Compound 1 was obtained as light brown amorphous powder. The molecular formula of 1 was determined as C₃₆H₂₂O₁₈ from LREIMS, ¹H, ¹³C, and ¹³C DEPT spectrum data. The 1H NMR spectrum of compound 1 showed two AB₂ systems at d 5.75 (2H, J=2.2 Hz), and 5.80 (1H, J=2.2 Hz), and two singlet signals at d 6.05 (2H, s) and 6.09 (2H, s) as well as 12 phenolic OH protons at d 9.29 (2H, s), 9.16 (4H, s), 9.15 (2H, s), 9.09 (2H, s), and 8.65 (2H, s), respectively.

The ¹³C NMR spectrum revealed the presence of 10 unsubstituted and 24 O-bearing aromatic carbons, and as well as two quaternary aromatic carbon signals. The molecular weight of compound 1 was twice more than bieckol by only two phloroglucinol unit differences excepting for two protons loss due to the direct linkage (742 vs 372). These data described above clearly indicated that compound 1 was composed of six phloroglucinol units. On the basis of the above data and an exact comparison with those values in the literature, compound 1 was assigned to be 6,6′-bieckol successfully and undoubtedly. The 6.4 ppm difference of ¹³C chemical shift value at C-8 between 6,6′-bieckol and 8,8′-bieckol led to the clear classification of both similar phlorotannins.

Compound 1: light brown powder (lyophilized); ¹H NMR (DMSO-d6, 400 MHz) d 9.29 (1H, s, OH-9), 9.16 (2H, s, OH-3′, 5′), 9.15 (1H, s, OH-2), 9.09 (1H, s, OH-4), 8.65 (1H, s, OH-7), 6.09 (1H, s, H-3), 6.05 (1H, s, H-8), 5.80 (1H, d, J=2.2 Hz, H-4′), 5.75 (2H, d, J=2.2 Hz, H-2′, 6′); ¹³C NMR (DMSO-d6, 100 MHz) d 123.5 (s, C-1)), 145.4 (s, C-2), 97.7 (d, C-3), 141.4 (s, C-4) 121.9 (s, C-4a), 141.3 (s, C-5a), 99.7 (d, C-6), 151.3 (s, C-7), 97.8 (d, C-8), 144.5 (s, C-9), 122.7 (s, C-9a), 137.2 (s, C-10a), 160.4 (s, C-1′), 93.7 (d, C-2′), 158.8 (s, C-3′), 96.1 (d, C-4′), 158.8 (s, C-5′), 93.7 (d, C-6′); LREIMS m/z 742.10 [M]+.

### Example 2

### Cell Viability Assay

H9 and H9/HIV-1IIIB cell lines were obtained through American Type of Culture Collection (Manassas, Va., USA). CEM-SS cell line from Dr. P. Nara and C8166 cell line from Dr. G. Farrar were provided by the EU Programme EVA Centre for AIDS Reagents, NIBSC, UK. CEM-SS, C8166, H9 and H9/HIV-1IIIB cell lines were grown in RPMI 1640 medium supplemented with 10% FBS, 100 μg of streptomycin per ml and 100 U of penicillin per ml. HIV-1IIIB virus stock was obtained from the culture supernatant of chronically infected H9/HIV-1IIIB cells. Cell-free virus was harvested from the supernatants by centrifugation and filtration through 0.22 μM filter. The virus stocks were stored as small aliquots at 80° C. until use.

The cytotoxic concentrations of 6,6′-bieckol were determined by MTT assay, a method based on the reduction of 3-(4,5-dimethylthiazol-2-yl)-2,5-diphenyltetrazolium bromide (MTT). Four hundreds of microliters of medium containing CEM-SS or H9 cells were cultured into a 48-well plate at a density of 10⁵ cells/ml. The plate was incubated overnight and treated with 100 μl of RPMI 1640 medium with/without 6,6′-bieckol. After 72 h of incubation, 100 μl of 500 μg/ml MTT was added to each well and the plate was incubated for another 4 h at 37° C. The blue formazan was dissolved in acidified propanol containing 50% DMSO and 4% Triton X-100. Optical density was measured at 540 nm with a microplate reader. The optical density of formazan formed by untreated cells was taken as 100% of viability.

### Example 3

### Measurement of Anti-HIV Activity

### Example 3-1

### Inhibition of Syncytia Formation

1×10⁵ C8166 cells in aliquots of 300 μl were seeded in triplicate to a 48-well plate containing 100 μl of serial dilutions of compound in complete medium. After 2 h of incubation, the cells were infected with 100 μl of stock supernatant of HIV-1IIIB diluted in complete medium at 200 CCID₅₀. The plates were incubated at 37° C. for 72 h and the number of syncytia was determined microscopically.

### Example 3-2

### Inhibition of Lytic Effects of HIV-1

In order to determine the anti-HIV-1 activity of 6,6′-bieckol on acutely infected CEM-SS cells, an MTT-formazan-based assay was used. Cells in log-growth phase were washed and resuspended in complete medium, and a 300 μl aliquot containing 1×10⁵ cells was added in triplicate to the wells of a 48-well plate containing the dilutions of compound in a volume of 100 μl of medium. Stock supernatants of HIV-1IIIB were diluted in complete medium to yield sufficient cytopathicity (90% cell kill in 7 days), and a 100 μl aliquot was added to the wells. Plates were incubated for 7 days at 37° C. and at the end of 7 days; cell viability was determined by MTT method as described before.

### Example 4

### Western Blot Analysis

1×10⁶ cells/ml H9 cells were cultured in cell culture plates. Following sample treatment, plates were incubated for 2 h and infected with HIV-1IIIB at 200 CCID₅₀. After 96 h of incubation at 37° C., cells were pelleted at 1000 rpm for 10 min and supernatant was harvested. The cells were washed 3 times with PBS and lysed with 500 μl of lysis buffer containing 50 mM Tris-HCl (pH 7.5), 0.4% Nonidet P-40, 120 mM NaCl, 1.5 mM MgCl₂, 2 mM phenylmethylsulfonyl fluoride, 80 μg/ml leupeptin, 3 mM NaF, and 1 mM DTT. Hundred micrograms of total protein was used for immunoblot analysis.

Culture supernatant containing virus was filtered through 0.22 μm filter, mixed with 30% polyethylene glycol (PEG) (50% v/v) with 0.4 M NaCl, and virus particles were pelleted at 15000 rpm for 45 min. The viral pellets were lysed, resuspended in SDS sample buffer and equal volume of viral lysates (20 μl) was loaded onto SDS gel. The proteins were subjected to denaturating SDSAGE in 25 mM Tris, 192 mM glycine, 0.1% SDS with a 4% stacking, and 10% separating gel. Separated proteins were transferred onto a nitrocellulose membrane, blocked in Tris buffered saline containing 0.1% (v/v) Tween 20 (TBS-T) and 5% skim milk powder. The membrane was probed with mouse anti-p24 monoclonal antibody (1:500, SantaCruz) and horseradish peroxidase (HRP)-conjugated anti-mouse IgG secondary antibody (1:5000, SantaCruz). The proteins were visualized by chemiluminescence (Fujifilm Life Science, Tokyo, Japan).

### Example 5

### Reverse Transcriptase Activity Assay

The activity of HIV-1 reverse transcriptase in the viral lysate was evaluated using a fluorescence RT assay kit (InvitroGen) according to the manufacturer's protocol. Briefly, 20 μl of reaction mixture containing a template/primer hybrid, poly(A)/d(T)16, and dTTP as a triphosphate substrate was added to the wells of amicrotiter plate and mixed with 5 μl of viral lysate containing various concentrations of 6,6′-bieckol. After incubation at 37° C. for 1 h, the reaction was stopped by the addition of 2 μl of 200 mM EDTA to each reaction. Fluorescence intensity was measured at 480 nm (excitation) and 520 nm (emission) with a GENiosmicroplate reader (Tecan Austria GmbH, Austria) after the addition of 173 μl of fluorescent PicoGreenreagent, which selectively binds to dsDNA or DNA-RNA heteroduplexes over single-stranded nucleic acids or free nucleotides.

### Example 6

### p24 ELISA

H9 cells (3×10⁶ cells/ml) were incubated in the presence or absence of HIV-1IIIB for 1 h at 37° C. Cells were washed to remove unbound viruses and resuspended at 3×10⁵ cells/ml in culture medium. Aliquots of 1 ml were placed in a 24-well culture plate containing an equal volume of medium with/without 6,6′-bieckol.

AZT was treated as positive control. In order to determine the amount of virus released to the medium, HIV-1 p24 antigen capture ELISA was carried out with a commercial kit (Perkin-Elmer Life Sciences, Boston, Mass.) according to the manufacturer's instructions.

### Example 7

### Co-Cultivation Assay

3×10⁴ C8166 cells were pre-treated with various concentrations of 6,6′-bieckol for 2 h and co-cultured with 3×10³ H9 cells chronically infected with HIV-1IIIB at 37° C. in a humidified atmosphere of 5% CO₂. After 72 h of incubation, the number of syncytia formed was counted using a microscope.

We obtained the following results through the examples 2 to 7.

6,6′-Bieckol exhibited no cytotoxicity up to 500 μM and the CC50 value of 6,6′-bieckol was calculated as 484 1M (FIG. 1A). HIV-1 induced cytopathic effect was measured by quantification of syncytia and the lytic effect of virus on C8166 and CEM-SS cells, respectively. 6,6′-bieckol inhibited HIV-1 induced syncytia formation and protected the cells from lytic effect of virus in a dosedependent manner. EC₅₀ of 6,6′-bieckol on the inhibition of the HIV-1 induced syncytia formation was 1.72 μM (FIG. 1B). With the highest concentration of 6,6′-bieckol (25 μM), 88% of syncytia formation was inhibited. 6,6′-bieckol protected the cells from HIV-1 induced lytic effects in vitro. At highest concentration of 6,6′-bieckol treated (25 1M), the protection of infected cells was more than 96% (FIG. 1C). The therapeutic index (TI) of 6,6′-bieckol was around 393. Inhibition of HIV-1 induced cytopathic effect was parallel with that of p24 antigen production. EC₅₀ of 6,6′-bieckol on inhibiting HIV-1 p24 antigen production was 1.26 1M (FIG. 1D).

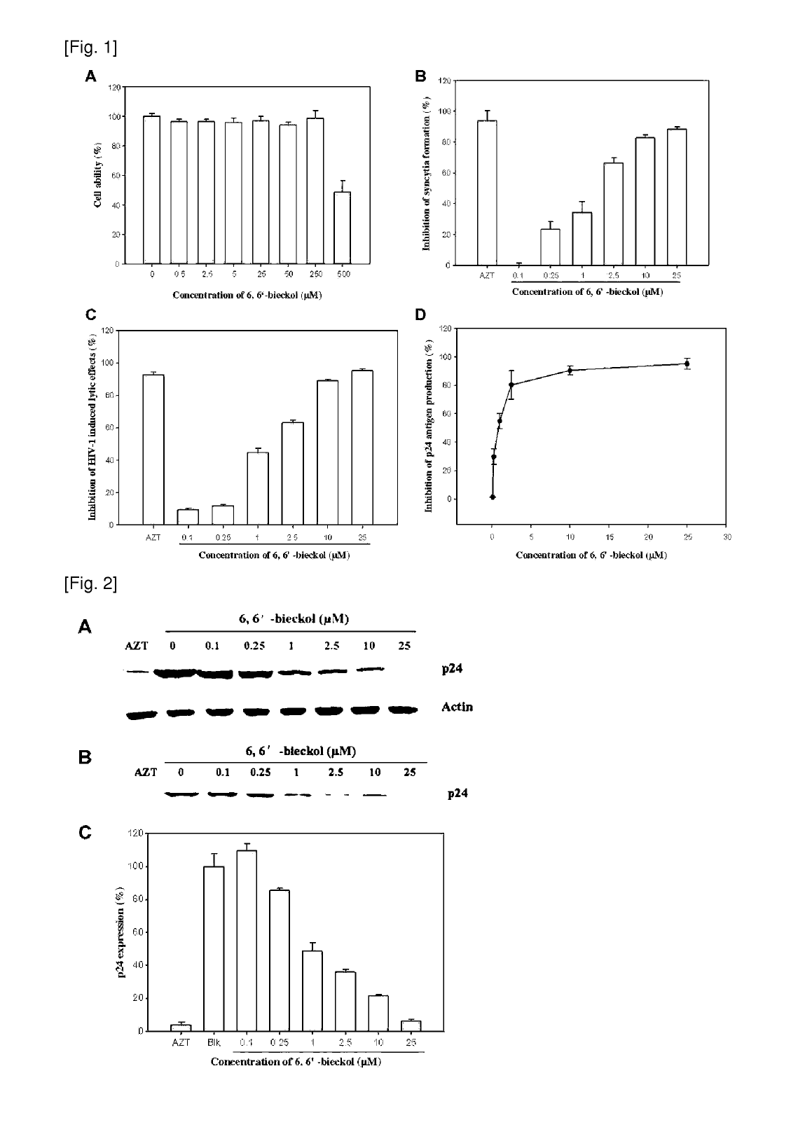

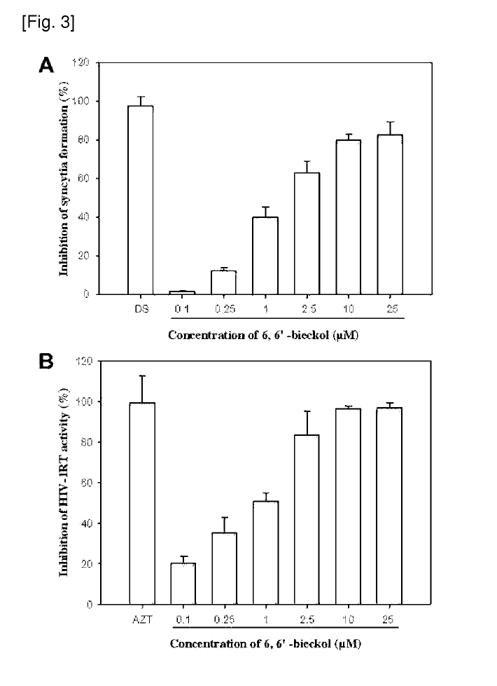

The inhibitory effects of 6,6′-bieckol on HIV-1 p24 antigen production were further determined by Western blot analysis of cell and culture supernatant (FIG. 2A). HIV-1 RT enzyme from cell culture supernatant was strongly inhibited by 6,6′-bieckol at relatively low concentrations (50.46% inhibition at 1 μM). 96.33% of HIV-1 RT enzyme activity was inhibited by 6,6′-bieckol at 10 1M (FIG. 1C). The fusion of virus or HIV infected cells with uninfected target cells is a critical step in HIV infection. According to data obtained from co-cultivation of C8166 cells with H9 cells chronically infected with HIV-1IIIB, 6,6′-bieckol inhibited the entry of HIV-1. 6,6′-bieckol inhibited cellvirus (FIG. 1B) and cell fusion (FIG. 3A). At the highest concentration of 6,6′-bieckol treated, the inhibition of syncytia formation was around 80% as a result of co-cultivation assay.

In the present invention, the anti-HIV-1 activity of 6,6′-bieckol was investigated. 6,6′-bieckol was shown to inhibit the cytopathic effects of HIV-1 on CEM-SS and C8166 cells as well as the activity of HIV-1 reverse transcriptase enzyme activity in vitro. Phloroglucinol derivatives mostly exhibit HIV-1 inhibitory activity by blocking the interaction between RT and RNA template. This type of inhibition is similar to those of some flavonoids. Kilkuskie et al. 19 reported that among the tannins they tested; most of them were cytotoxic at the effective concentration, which means therapeutic indices (TI) of the compounds were around 1. They evaluated the HIV replication and reverse transcriptase inhibitory activities of a variety of tannins including gallotannins, ellagitannins, condensed and complex tannins in order to investigate the correlation between HIV inhibitory activity and RT inhibitory activity of tannins. The results indicated that reverse transcriptase inhibition did not correlate with the inhibition of HIV replication. As demonstrated in FIGS. 1B and 4B, data obtained from HIV-1 induced cytopathic effects and RT activity studies indicated that HIV-1 replication inhibition correlated with the inhibition of reverse transcriptase. 6,6′-bieckol inhibited both HIV-1 induced lytic effect and HIV-1 RT activity almost completely. These data reveal that unlike other tannins, 6,6′-bieckol is non-cytotoxic at concentrations which inhibit HIV-1 replication and reverse transcriptase activity effectively.

Entry step of HIV-1 is an attractive target for new generation drug candidates. Therefore, the inhibitory effect of 6,6′-bieckol on HIV-1 entry was investigated. Nonaka et al. claimed that a variety of tannins inhibited the replication of HIV, probably by interfering with HIV-cell interactions. 6,6′-bieckol inhibited the HIV-1 induced syncytia formation as a result of data obtained from co-cultivation of uninfected C8166 cells with H9/HIV-1IIIB cells. Tannins show their inhibitory activity on HIV-1 entry by inhibiting gp41 six-helix bundle formation. However, when entry inhibition profile of 6,6′-bieckol is compared with the inhibition of cytopathic effects, it could be concluded that HIV-1 induced cytopathic effect is not only reduced by the inhibition of entry, but also by inhibition of HIV-1 reverse transcriptase enzyme activity subsequently. p24 antigen production was determined with p24 antigen capture ELISA and Western blot analysis. 6,6′-bieckol inhibited the production of p24 antigen more than 90% at 10 1M supported by data obtained from ELISA (FIG. 1D). As shown in FIG. 2C, the inhibition of p24 antigen production at the concentration of 25 1M of 6,6′-bieckol was comparable to that of AZT (5 μM). 8,8′-bieckol, which is structurally similar to 6,6′-bieckol, inhibited HIV-1 recombinant RT and protease activity with IC₅₀ s of 0.51 μM and 81.5 μM, respectively. Reverse transcriptase inhibitory activity of 6,6′-bieckol was consistent with those of 8,8′-bieckol. According to Western blot analysis, no or weak bands were detected at the locations of p55 gag related proteins (p55, p41, and p24) except for p24, which indicated that 6,6′-bieckol did not inhibit the protease. However, 6,6′-bieckol inhibited the p24 antigen production in a dose-dependent manner both in culture supernatant and cell (FIGS. 2A and B) by inhibiting the activity of reverse transcriptase. Generally, the structure-activity relationships should go to the unique skeleton with dibenzodioxin linkage, linkage positions of phloroglucinols, and phenolic groups exiting in 6,6′-bieckol.

In conclusion, 6,6′-bieckol is a novel safe compound of natural origin with significant HIV-1 replication and reverse transcriptase inhibitory activity. The present study suggests that 6,6′-bieckol might be a promising candidate for the design of novel HIV-1 RT inhibitors with its special structure.

1. A pharmaceutical composition having anti-HIV-1 inhibitory activity, containing phloroglucinol derivatives from Ecklonia cava as an effective ingredient.

2. The pharmaceutical composition according to claim 1, wherein the phloroglucinol derivative is 6,6′-bieckol of formula 1.

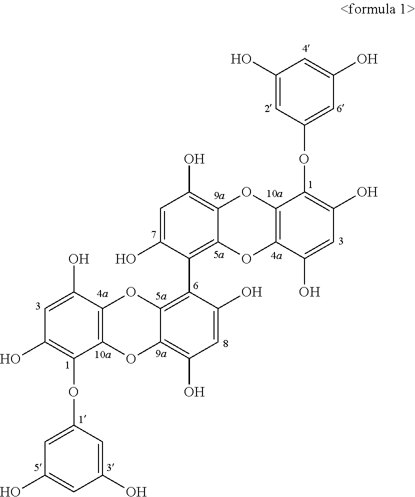

3. A health functional food having anti-HIV-1 inhibitory activity, containing phloroglucinol derivatives from Ecklonia cava as an effective ingredient.
